(19) United States
(12) Patent Application Publication
(10) Pub. No.: US 2006/0276727 A1
(43) Pub. Date: Dec. 7, 2006

(54) Software for continued evaluation, diagnosis and monitoring of patients on physical therapy

(76) Inventor: Tim Terrio, Bakersfield, CA (US)

Correspondence Address: ZUBER & TAILLIEU, 9th FLOOR, 9595 WILSHIRE BLVD., BEVERLY HILLS, CA, 90212, US

(21) Appl. No.: 11/141,719

(22) Filed: Jun. 1, 2005

(51) Int. Cl. A61B 5/02; A61B 5/103

(52) U.S. Cl. 600/595; 600/487

(57) ABSTRACT

A computer-program-implemented system for creating and maintaining physical therapy patient information and displaying the patient information via a graphical user interface comprised of a series of pages which are interconnected/interlinked. The computer-program-implemented system includes the ability to efficiently evaluate therapy patients in a standardized fashion by receiving, recording and storing patient demographic information in a database, selecting a standardized automated evaluation from a previously generated list of several possible standardized automated evaluations (the selected standardized automated evaluation containing a series of questions), recording responses to said series of questions contained in the selected standardized automated evaluation and storing said responses in said database, and assigning a numerical value or score to said evaluation as a function of said recorded and stored responses.

### FIELD OF THE INVENTION

The invention relates generally to software utilized in the medical industry. More particularly, the invention relates to software specifically designed to be used in the continued evaluation, diagnosis and monitoring of patients in physical therapy.

### BACKGROUND OF THE INVENTION

Medicine and the practice thereof has become extremely advanced as new techniques and technologies for providing improved health care and customer service have developed. Unfortunately, not all medicine is practiced in a uniform fashion. Doctors, nurses and physical therapists are all human and their individual medical background and methodology can vary from one extreme to another, depending upon their education, training and experience.

For example, if a patient comes to a doctor with a physical problem or ailment involving his or her joints or bones, the doctor may prescribe physical therapy for the patient. Often this may occur after some type of surgical procedure is performed; although surgery is not always a necessary precursor to physical therapy and the doctor may prescribe therapy in lieu of surgery.

In recent years, it has become necessary for the physical therapist to demonstrate “medical necessity” of the physical therapy treatments in order for the therapist to be reimbursed for professional services. In order to demonstrate this medical necessity it is important to first establish the patient's current level of function, loss of range of motion, decreases in strength, loss of balance etc. The therapist must then set a treatment plan and begin treatments. When a patient is first referred to a therapist, he or she will usually be given an evaluation in order to ascertain his or her degree of pain along with any physical dexterity or ambulatory limitations. For example, if a patient is referred to a physical therapist for a problem with his or her hip joint, the patient may be evaluated to ascertain his or her ability to perform basic hip adduction and hip abduction movements, as well as to ascertain his or her overall degree of comfort and/or pain in performing such movements.

Based upon this evaluation, and the doctor's recommendations, the therapist will usually provide a therapy regimen for the patient to perform. The regimen may consist of a number of exercises and/or physical movements, use of modalities to facilitate motion or decrease pain, and/or mobilization and stretching techniques which the patient must perform at the therapist's office, or at home, on a regular schedule.

State laws and practice acts require the physical therapist to continually monitor the patient's response to therapy and any improvements or regressions from the original state. “Medical necessity” is demonstrated when the patient continues to improve functionally through improved/increased range of motion, strength, improved ambulation/balance, etc. In order to document these improvements, re-assessments must be performed on a regular basis. Accordingly, after several weeks, the patient may then be re-evaluated in order to ascertain his or her progress on the proscribed therapy regimen. Usually, an evaluation very similar, if not identical, to the original evaluation is once again performed and the results are compared with the previous results in order to ascertain whether the patient is improving.

One problem with this process is that it lacks uniformity. More specifically, depending upon the therapist involved and his or her individual training, certain evaluations may or may not be performed. There are literally hundreds of specialty tests in physical therapy, many with varying degrees of specificity and sensitivity. Depending on the therapist's education and/or experience, they will utilize only a portion of these tests. Unless each therapist reads medical literature constantly, they may not know which tests have been shown to have the best specificity and sensitivity for a given problem. Accordingly, while most therapists are apt to perform several basic evaluations with a high degree of uniformity, many therapists may not perform more advanced evaluations, which might allow the therapist to more accurately measure the patient's physical limitations and/or degrees of pain in all situations. Therefore, the quality of the evaluation and the resultant proscribed therapy may not be as good as possible if the patient were more thoroughly evaluated.

Short of requiring every therapist in the country to maintain current and ongoing academic continued education and providing intense monitoring of every therapist to ensure he or she is performing at the top level, there are no known ways for standardizing the therapy process.

It is desireable to have at least some type of automated and easy to use system which might assist to streamline and standardize the physical therapy process such that patients are evaluated in a consistent and complete fashion, regardless of the experience or education of the particular therapist involved. It is also desireable to have patient information and the results of each such evaluation recorded, stored and displayed in a meaningful and easy to read fashion such that the progress of a patient can be easily monitored throughout the therapy process.

### SUMMARY OF THE INVENTION

The invention is directed toward software used in physical therapy which will assist therapists in providing fully standardized, thorough evaluations and continued monitoring of physical therapy patients. According to one aspect of the invention, a computer-program-implemented system for creating and maintaining a patient-doctor-therapist data base is described which includes graphic means for receiving patient information, providing patient evaluations, and displaying the patient information and evaluation results as a series of webpages which are interconnected/interlinked. The information is stored in a database and is called for display as a user navigates through the system.

According to still another aspect of the invention, the system further includes a multiplicity of authorization levels, wherein users of the system are assigned a user type, with each user type having a different level of authorization, such that some users have more restrictions than others. A login or authentication process is used in order to identify each user, after which such use may only obtain and/or enter certain information dependent upon his or her authorization.

In addition, the computer-program-implemented system includes a graphical user interface for providing detailed patient information for viewing by a Doctor/Physician, a Physical Therapist, and an Administrator. The graphical user interface creates webpages which include specific patient/referral information. The graphical user interface also allows Physical Therapist users to conduct standardized/uniform patient evaluations over the course of therapy and monitor the continuing results of those evaluations.

In a preferred embodiment, the graphical user interface is capable of providing automated, specific and proprietary evaluations that can be documented, recorded and stored in a database using the software of the present invention. Each evaluation has its own particular EVALUATION PAGE which contains specific requested information pertinent to that evaluation. In a preferred embodiment, evaluations may be functional and non-functional. Functional evaluations are geared more toward evaluating a patient's self-subjective overall level of satisfaction with his or her progress. It will inquire into the patient's level of pain/comfort, level of activity and mobility, ability to perform every day tasks and skills with little or no pain/discomfort, and the like. In this way a functional evaluation is based very strongly on a patient's subjective responses to a series of questions designed to ascertain whether the patient is improving. Non-functional evaluations will instruct a Physical Therapist to make certain in office, observations and/or measurements related to the degrees of mobility, range of motion, strength, palpation and special tests.

Accordingly, each particular EVALUATION PAGE contains a series of questions and/or objective measurements/evaluations which are required to be completed by the therapist conducting the evaluation. The questions and required objective measurements/evaluations have been compiled from an amalgam of various medical literature and medical information available and include several proprietary questions and objective measurements/evaluations, all of which have been specifically tailored and selected in order to standardize and streamline the evaluation process such that evaluations for any one particular ailment or injury can be uniform and will be independent of the particular level of skill, education and experience of the therapist/user.

In addition, the software of the present invention includes methods for evaluating, scoring and storing functional outcomes from functional evaluations. As explained above, in a preferred embodiment the software includes both functional and non-functional evaluations (with functional evaluations being directed more toward obtaining objective information about the patient's condition, such as his or her level/degree of pain, level of activity, level of energy, and/or ease in performing every day activities). The functional evaluations are assigned a functional outcome score when they are completed and these scores are tracked over time in order to ascertain whether the patient is improving functionally in their every day life.

Additionally, the software of the present invention also generates a range of motion value based upon specific mobility and range of motion measured by the Physical Therapist when conducting a non-functional evaluation. As explained earlier, non-functional evaluations measure a patient's actual objective characteristics or condition, and may be very detailed or more basic. Non-functional evaluations require a Physical Therapist to make certain, in office observations/measurements regarding the patient's mobility and range of motion. In a preferred embodiment, the present invention will utilize a summation of specific active range of motions for each specific joint evaluated, in order to come up with a numerical range of motion value. This numerical range of motion value will be graphed separately from the automated scores assigned to functional outcomes of functional evaluations. Accordingly, the goal of the therapy is to see a linear improvement in both scales (active range of motion and functional outcome scores). If both graphs are trending up then the patient is improving in mobility and range of motion, and is recognizing and responding to this improvement through improved functioning in their every day life. If both values are trending down, then the patient is getting worse and is not responding to the current therapy treatment. If one scale is trending up and the other is trending down (if for example the numerical range of motion value is improving but the overall functional outcome score is not improving) then more investigation is warranted in order to determine why the patient is reporting something different than the objective measurement of active range of motion (i.e. why the patient is not improving functionally in their every day life activity).

In short, the goal of therapy is to continually increase or improve upon a patient's overall score on both scales. If the overall numerical value or score assigned to each functional outcome continues to increase or improve over the course of the therapy, as continued functional evaluations are performed, then the patient is responsive to the proscribed therapy and is noticeably improving. If, however, the overall numerical value or score assigned to each functional outcome decreases or remains fairly constant over the course of the therapy, as continued functional evaluations are performed, then the patient is non-responsive to the proscribed therapy and is not improving, thereby signaling to the therapist that a deeper problem may exist. Accordingly, the therapist can then discuss the issue with the patient's referring physician and an alternate therapy program may be developed or the patient may be re-examined by the referring physician in order to better ascertain the reason for the continued injury/ailment.

Using the software, therapists will be better able to fully and completely evaluate new and existing patients, monitor their therapy progress, and proscribe or alter their physical therapy regime as needed. Use of the software will also facilitate broader and more meaningful communication between the physical therapist, the doctor and the patient as the patient is continually evaluated. Physicians will have access to simple, easy to read documentation which shows if their patients are improving, staying the same or getting worse. Additionally, the use of standardized evaluations can further facilitate clinical research in order to define which treatments are related to the best outcomes for patients.

### DESCRIPTION OF THE FIGURES

Other features and advantages of this invention will become more apparent in the following detailed description of the preferred embodiment of this invention, with reference to the accompanying drawings, in which:

FIG. 1 contains a flow chart which illustrates this LOGIN PROCESS, such that an authorized user may then gain access to proprietary and confidential patient information and use the software of the present invention to evaluate and monitor such patients;

FIG. 2a-2c each illustrate various versions of a MAIN MENU page as seen through the user interface when using the software of the present invention in accordance with a first preferred embodiment;

FIGS. 3a and 3b each provide a preferred embodiment for a PATIENT RECORDS MENU which is displayed via the graphical user interface using the computer-program-implemented system of the present invention;

FIG. 4 illustrates a flow chart which shows these two different alternatives for accessing particular patient information;

FIG. 5 illustrates a PATIENT DETAILS page seen through the user interface when using the software of the present invention in accordance with a first preferred embodiment;

FIG. 6 illustrates a REFERRAL DETAILS page as seen through the graphical user interface when using the computer-software-implemented system of the present invention in accordance with a first preferred embodiment;

FIG. 7 illustrates a flow chart which shows the steps performed in conducting a new evaluation in accordance with a preferred embodiment of the present invention;

FIG. 8 illustrates a sample FUNCTIONAL OUTCOME for conducting a functional evaluation of an ankle injury in accordance with a preferred embodiment of the present invention; and

FIG. 9 illustrates a sample EVALUATIONS PAGE for conducting a detailed evaluation, including objective measurements for range of motion, of an ankle injury in accordance with a preferred embodiment of the present invention.

### DETAILED DESCRIPTION OF PREFERRED EMBODIMENTS

The invention is directed toward a computer-program-implemented system used in physical therapy which will assist therapists in providing fully standardized, thorough evaluations and continued efficient monitoring of physical therapy patients. Using the system and software of the present invention, therapists will be better able to fully and completely evaluate new and existing patients, monitor their therapy progress, and proscribe or alter their physical therapy regime as needed.

The invention includes a computer-program-implemented system for creating and maintaining a patient-doctor-therapist database which receives and stores patient demographic information and patient evaluation information. In a preferred embodiment the invention includes a graphical user interface through which various patient/medical information is retrieved/requested to be entered. The information is then organized and displayed via the graphical user interface as a series of webpages which are interconnected/interlinked, such that a user may move through the information from page to page. Preferably, all of the information is stored in a database and is called for display and integration into the graphical user interface as a user navigates through the system. Accordingly, each webpage to be displayed is actually designed as a template which is then created or completed “on the fly”, by calling certain information from the database and then displaying it in a clear, legible and easy to understand format on the templated page.

Login Overview

As with many proprietary software applications, the software of the present invention includes a built-in security measure referred to as a “LOGIN PROCESS” such that patient information remains protected and confidential, and can only be viewed by authorized personnel. Accordingly, the computer-program-implemented system of the present invention preferably includes a multiplicity of authorization levels, wherein users of the system are assigned a user type, with each user type having a different level of authorization, such that some users have more restrictions than others as to what information will be made available to such user. A login or authentication process is used in order to identify each user, after which such user may only obtain and/or enter certain information dependent upon his or her authorization.

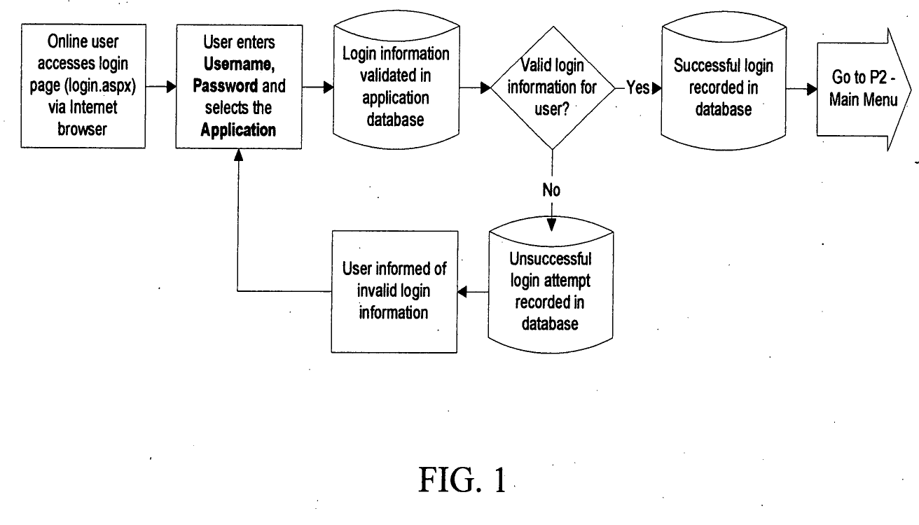

FIG. 1 contains a flow chart which illustrates this LOGIN PROCESS, such that an authorized user may then gain access to proprietary and confidential patient information and use the software of the present invention to evaluate and monitor such patients. As shown, when the software is first initialized, a LOGIN page is displayed through the software interface. The LOGIN page contains appropriate prompts for a new user login. The user will login using a proprietary Username and Password which has been assigned to that user. The information entered by the new user is then validated. If the information is recognized then the login process has been successful and the software interface is refreshed to display a MAIN MENU page. If the information is not recognized then the login process has failed. The failed login attempt is recorded and the user is once again prompted to attempt a new login with a correct Username and/or Password.

In a preferred embodiment, a user is defined by user type. Preferably, the invention includes at least three different use types including a Doctor/Physician, a Physical Therapist, or an Administrator. It is understood that these user type definitions are intended for illustrative purposes only and the actual number and name of each possible user type may be different. For example, the software may be designed to allow for an additional user type for an outside insurance agent/adjuster to access individual patient information which can be used to process insurance claims much more quickly and efficiently.

The user type is defined by the login and basically limits the type of informational items which may be accessed when using the software. Accordingly, when a user logs in and his or her Username and Password are identified by the system, a user type is also identified for the user and the particular information which may be recorded and/or displayed as the user navigates through the system are dependent upon the user type.

Main Menu Overview

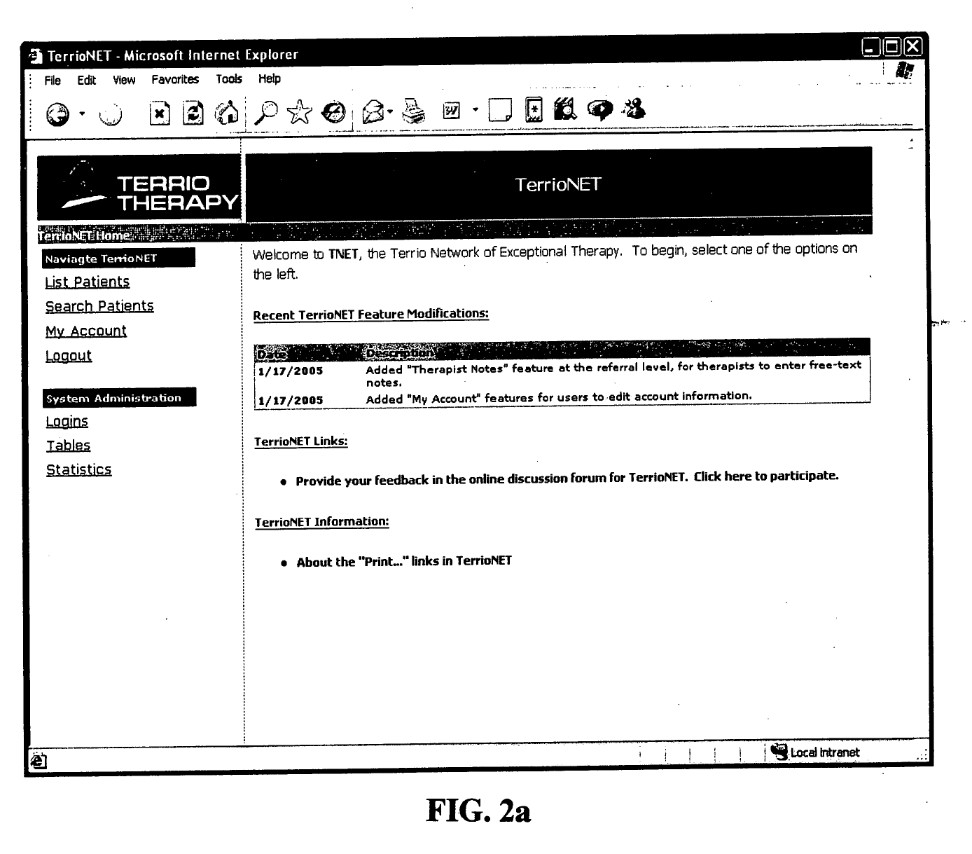

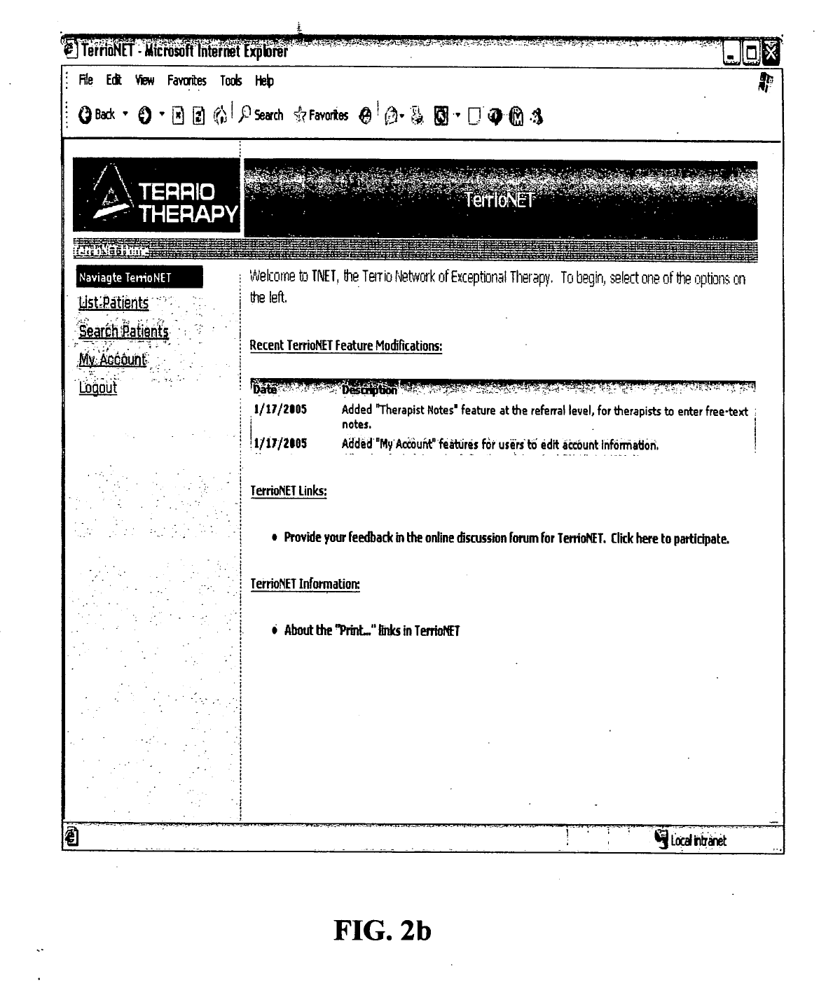

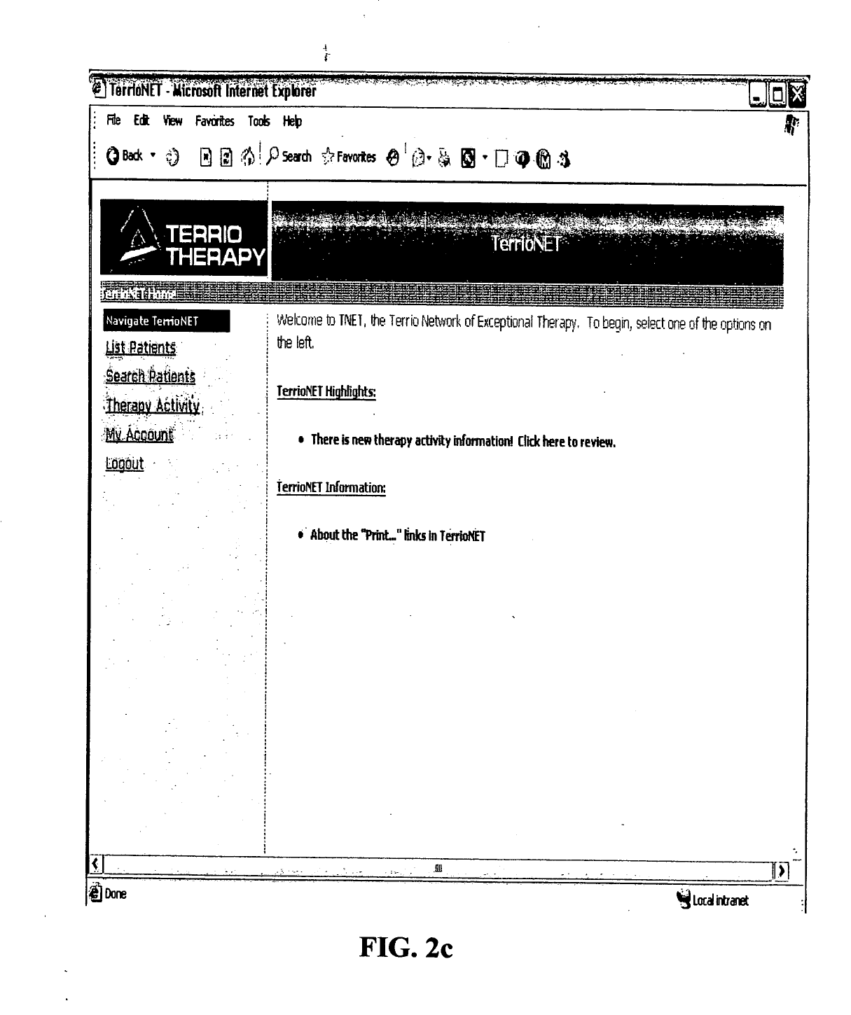

As explained earlier, in a preferred embodiment the invention includes a graphical user interface through which various patient/medical information is retrieved/requested to be entered. The information is preferably organized and displayed via the graphical user interface as a series of webpages which are interconnected/interlinked, such that a user may move through the information from page to page. The MAIN MENU page is the first page encountered after a successful login attempt when using the software of the present invention. FIGS. 2a-2c each illustrate variations of a MAIN MENU page as seen through the user interface when using the software of the present invention in accordance with a first preferred embodiment.

FIG. 2a illustrates a preferred embodiment for the MAIN MENU page as it would be visible to an Administrator. FIG. 2b shows a preferred embodiment of the MAIN MENU page as it would be visible to a Physical Therapist. FIG. 2c illustrates a preferred embodiment of the MAIN MENU page as it would be visible to a Doctor/Physician.

As shown in FIGS. 2a through 2c, the MAIN MENU page is preferable divided into two main sections. On the left hand side of the MAIN MENU page are a user's navigational options, which vary according to the type of user. As explained earlier, in a preferred embodiment of the present invention, a user is defined by user type. In a preferred embodiment, there are at least three user types including an Administrator, a Physical Therapist, or a Doctor/Physician. The user type is defined by the login and basically limits the type of informational items which may be accessed when using the software. For example, an Administrator would have the ability to modify display options, change menus and/or create additional webpages for viewing. Conversely, a Doctor/Physician would preferably have no ability to modify the program; but may be able to alter or add referral information and/or change the proscribed therapy.

Accordingly, when a user logs in and his or her Username and Password are identified by the system, a user type is also identified for the user and the items to be displayed in the navigational options of the MAIN MENU page are then selected and displayed on the left hand side. The particular items displayed in the navigational options of the MAIN MENU are dependent upon the user type. For example, the System Administrator has access to System Administration navigational options which are not displayed on a MAIN MENU page visible to a Physical Therapist or a Provider Staff.

On the right hand side is displayed instructional information and/or information appropriate to the current user. For example, in a preferred embodiment of the MAIN MENU page made visible to a Doctor/Physician (as shown in FIG. 2c), such a user will see two types of information, TerrioNET Highlights and TerrioNET Information. The TerrioNET Highlights are dynamic messages that are displayed based upon the particular user and some content in the database. One example of this type of TerrioNET Highlights information would be recent patient activity. Each time new activity is recorded for a particular patient, such as a new goal or new evaluation, this activity is recorded into an activity table in the database. A Doctor/Physician user that logs into the application who has access to the patient will see a notification that there is new activity for review, thus directing the Doctor/Physician to new information pertinent to the patient which may have recently been entered by the Physical Therapist. The TerrioNET Information messages are static links to any information that needs to be made available to a user, such as help with printing.

Patient Records Menu Overview

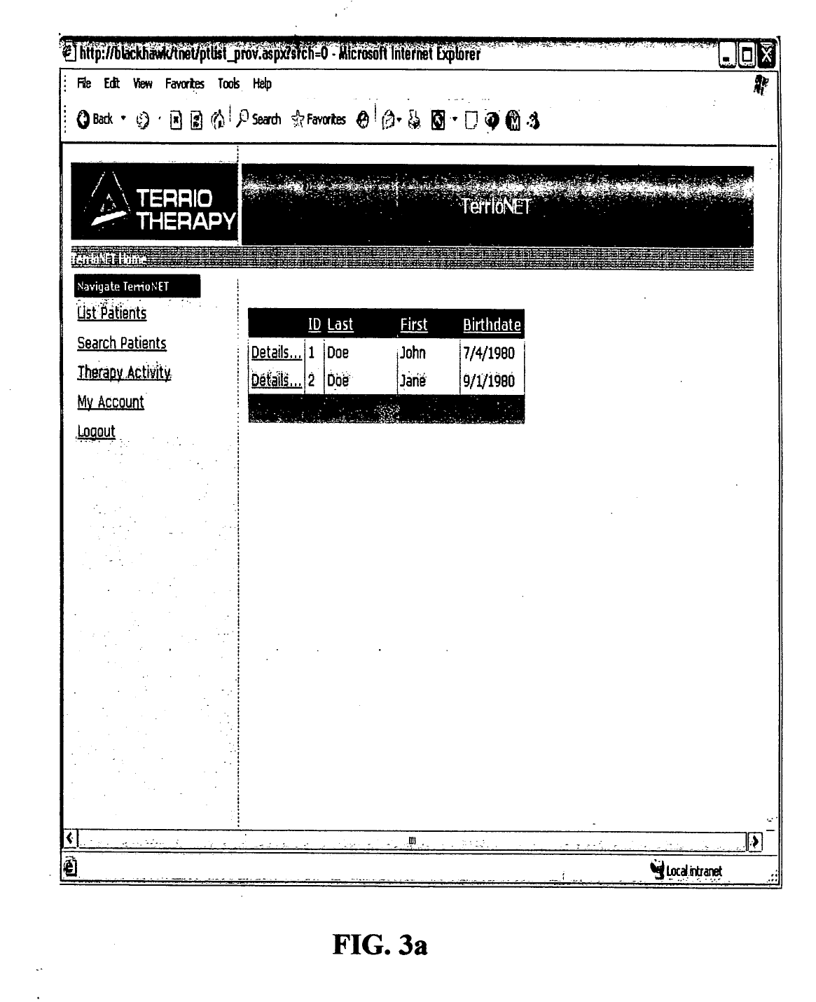

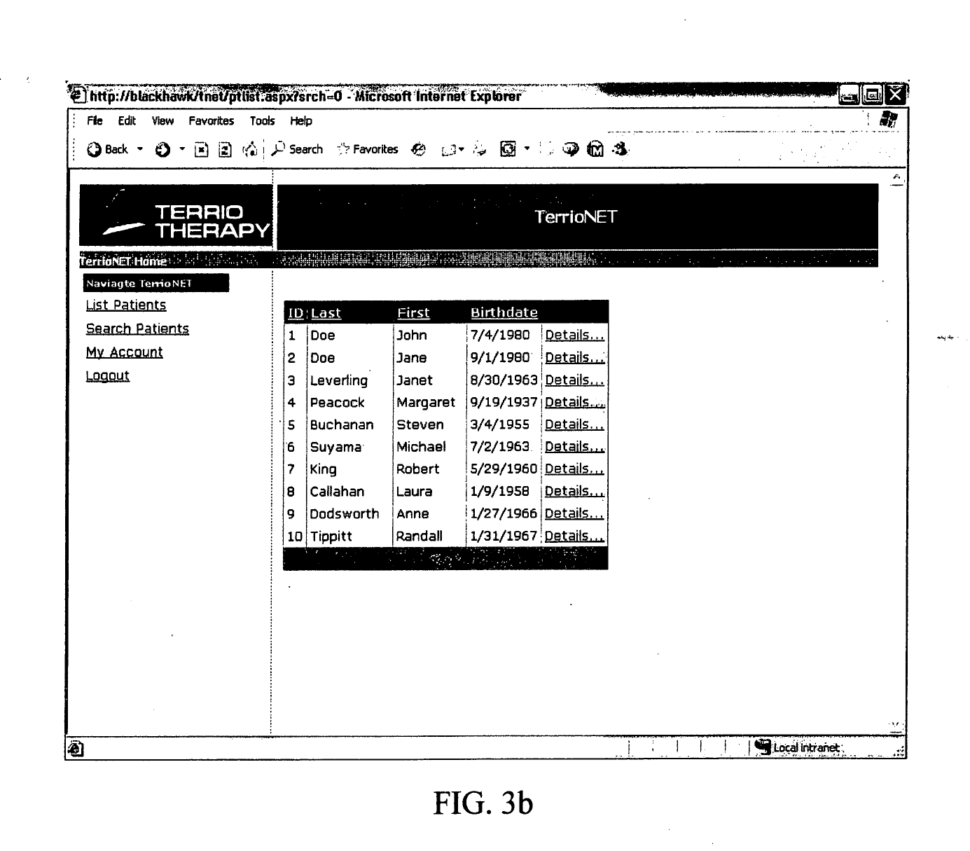

From the MAIN MENU, a user can preferably access particular patient information from a PATIENT RECORDS MENU in any one of two ways. FIGS. 3a and 3b each provide a preferred embodiment for a PATIENT RECORDS MENU which is displayed via the graphical user interface using the computer-program-implemented system of the present invention. More specifically, FIG. 3a illustrates a preferred embodiment for a PATIENT RECORDS MENU wherein patient information will be retrieved via a search, as further described hereinafter, and FIG. 3b illustrates a preferred embodiment for a PATIENTS RECORDS MENU wherein patient information will be retrieved by patient listing.

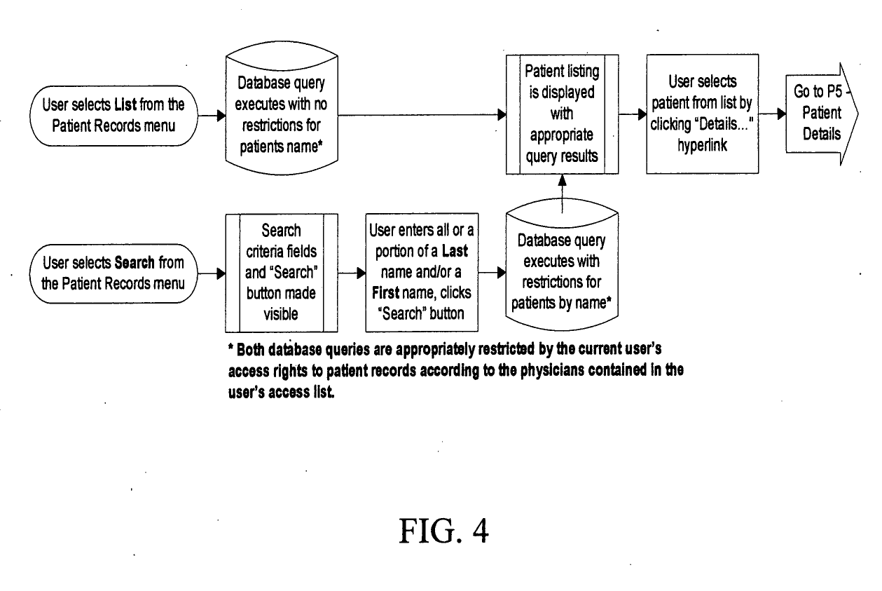

FIG. 4 illustrates a flow chart which shows these two different alternatives for accessing particular patient information. First, from the MAIN MENU a user may select a PATIENT LISTING navigational option, in which case the user will then be provided with a PATIENT RECORDS MENU listing of all the patients being monitored/treated by that particular user. In this option, the software searches a particular field in the patient information identifying the user and selects only those patients matching the particular user, thereafter displaying a selectable listing of such patients. The user may then scroll through the listing and then simply select the particular patient from such listing. Thus, for example, referring to FIG. 3b there is shown a list of patient names, in alphabetical order, for all patients being treated by the user. Accordingly, assuming a Physical Therapist using the software of the present invention wishes to see a listing of all patients he or she is treating, he or she would select the LIST PATIENTS navigational option on the left hand side of the MAIN MENU page (FIG. 2b) and then a list of all patients being treated by that Physical Therapist would be shown (FIG. 3b).

Alternatively, and in particular in the case of a large patient listing, the user may search for a particular name by selecting a PATIENT SEARCH navigational option in the MAIN MENU. Referring again to FIG. 4, when a user selects this option, he or she will be prompted with a Search Criteria field and a SEARCH Button. The user will then simply type in the patient's first and/or last name into the Search Criteria field and hit the SEARCH Button. In this option, the software searches a particular field in the patient information identifying the patient name. Preferably, because more than one patient may have the same last name, the software will also search a particular field in the patient information identifying the provider and select only those patients matching both the identified name and the particular user/provider, thereafter displaying a PATIENT RECORDS menu which contains a selectable listing of such patients. Referring to FIG. 3a, there is shown a listing of all patients having the last name DOE and being treated by said user of the system.

Once the user has either selected to have patients listed or searched, and the results have been retrieved, the user may then select the particular patient from the list provided, in order to obtain more information specific to that therapy patient. When the user selects a particular patient name from the selectable listing, a PATIENT DETAILS page is then displayed through the graphical user interface of the computer-software-implemented system of the present invention.

Patient Details Overview

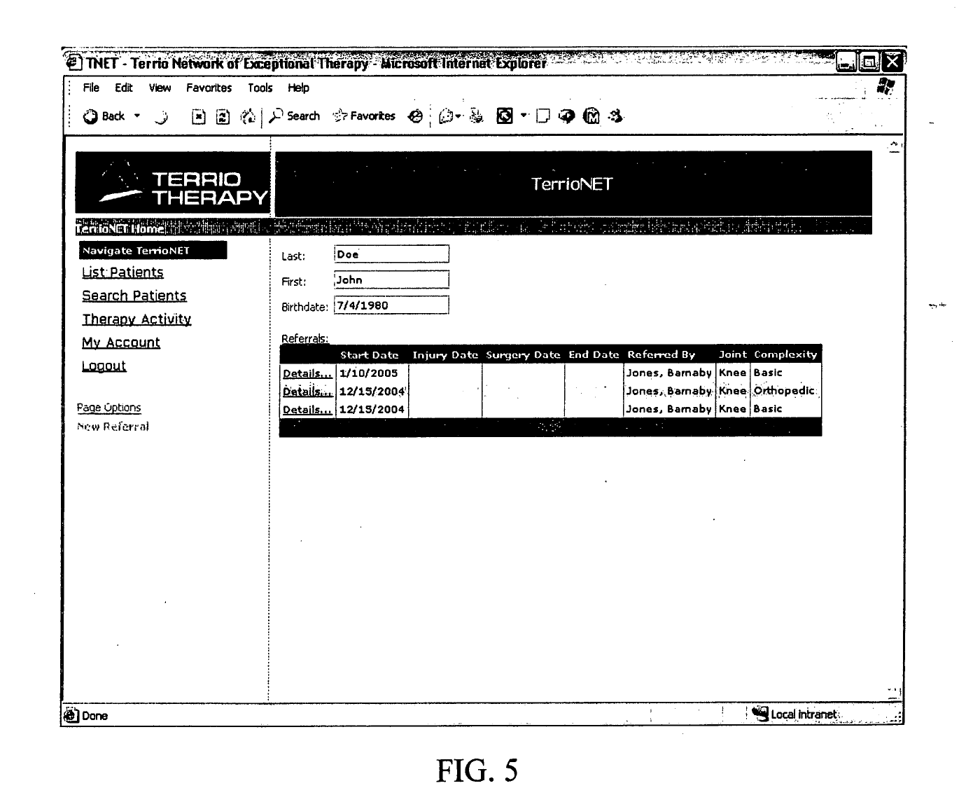

FIG. 5 illustrates a PATIENT DETAILS page seen through the graphical user interface when a Physical Therapist user utilizes the computer-software-implemented system of the present invention in accordance with a first preferred embodiment. As shown in FIG. 5, this particular PATIENT DETAILS page provides particular patient details and information relative to the patient named JOHN DOE and includes further navigational items which are user selectable by the Therapist user.

In general, the PATIENT DETAILS page will preferably include a series of Referral Listings which are displayed in reverse chronological order, with the last Referral Listing being at the top of the series. Referral listings basically provide information relevant to each patient referral to a particular Physical Therapist or facility over time. Thus, if a patient was referred to a facility for physical therapy surrounding a neck or back injury at one point in time and was then referred to the Physical Therapist or facility a second time, several years later, for a knee injury, then there will be at least two Referral Listings for that patient. Each Referral Listing will include basic patient demographic information pertinent to that referral (such as the patient's name, address, birthdate, therapy start date, injury date (if known), surgery date (where applicable), projected end date for the therapy (based upon the initial referral), the name of the referring physician, the particular bones or joints involved and the complexity of the evaluation). Complexity of the evaluation describes the complexity of the initial evaluation—i.e. the level of involvement of multiple joints, bones, etc. Some injuries are simple/limited, involving only one joint or bone, while others may be more complex (involving more that one area of the body). The complexity of the evaluation may be used for billing purposes and makes coordinating billing and therapy much easier. Accordingly, in a preferred embodiment evaluations are considered to be brief limited, intermediate, extended or comprehensive and are assigned such level of complexity accordingly.

Each Referral Listing in the series will also include a Referral link (labeled as Details . . . in FIG. 5) which links to a new page (a REFERRAL DETAILS page) containing more detailed information about that particular Referral Listing. Accordingly, if a patient had been referred to therapy for a neck injury at one point in time and then referred to therapy for a back injury at another, later point in time, then the Patient Details page would include at least two Referral Listings, with one Referral Listing having information about the back injury appearing at the top of the series and a second Referral Listing having information about the neck injury occurring later in the series, below the back injury.

As explained earlier, each Referral Listing preferably includes a Referral Link (labeled as Details . . . in FIG. 5). By selecting any of the Referral Links, the user is taken to a new page (a REFERRAL DETAILS page) which contains further detailed information about that particular referral. For example, referring to FIG. 5, one can see that patient John Doe was referred by physician Barnaby Jones for therapy related to a basic knee injury on Dec. 15, 2004 and the patient was again referred by physician Barnaby Jones for therapy related to a basic knee injury on Jan. 10, 2005. Each referral includes a link, which when selected will link to a REFERRAL DETAILS page wherein more specific information about that referral may be viewed by the Therapist user.

Referral Details Overview

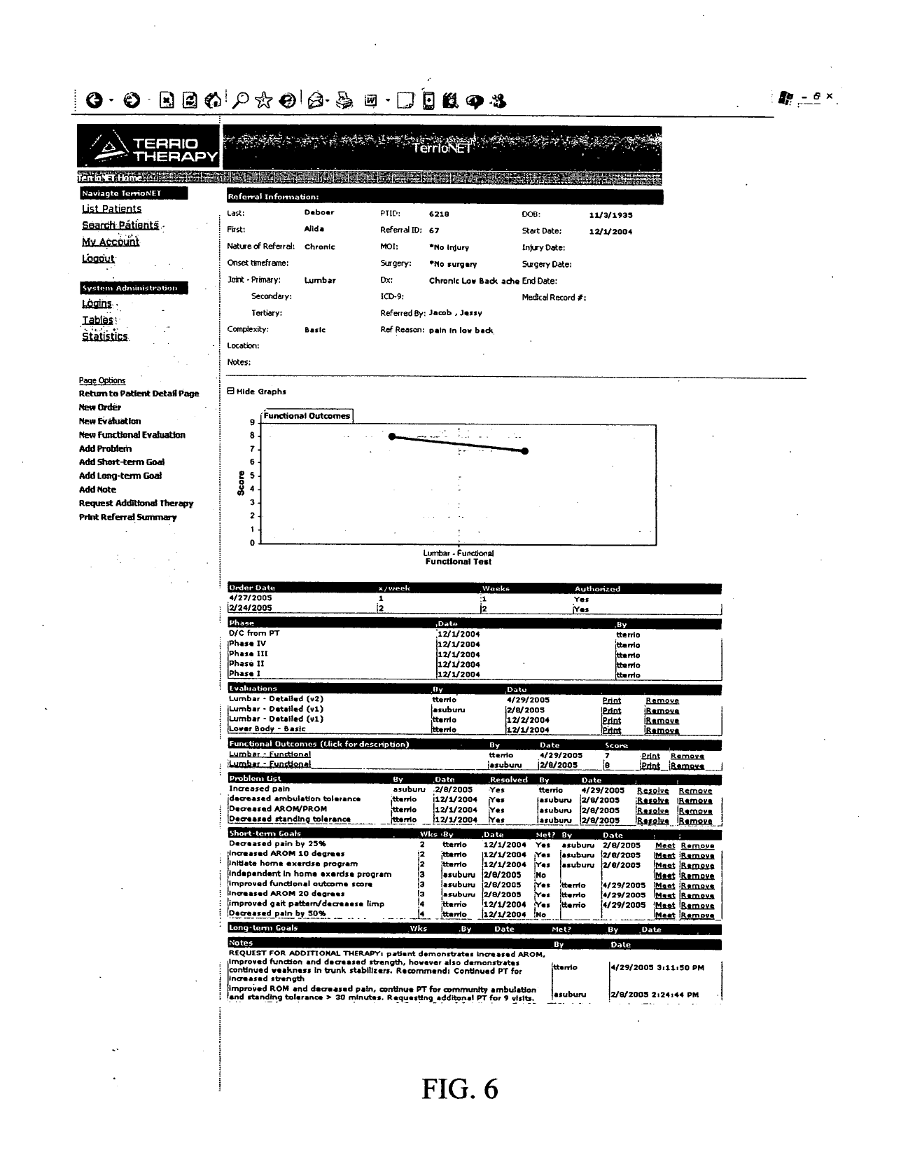

FIG. 6 illustrates a REFERRAL DETAILS page as seen through the graphical user interface when a Physical Therapist user utilizes the computer-software-implemented system of the present invention in accordance with a first preferred embodiment. As shown, the REFERRAL DETAILS page provides particular patient details and further navigational items which are user selectable. More specifically, the REFERRAL DETAILS page includes multiple data sets that relate to a specific therapy referral and provides further user selectable options for navigation and activity.

As shown in FIG. 6, the REFERRAL DETAILS page is preferably divided into four major areas. In a first area, located on the left hand side of the REFERRAL DETAILS page, appear navigational items from which the Physical Therapist user may select different items of action. For example, if a Physical Therapist user finds they want to look at a different REFERRAL DETAILS page, he or she may jump back or return to the previous PATIENT DETAILS page and select a different Referral Link from the series of Referral Listings, as described earlier herein. Alternatively, from the REFERRAL DETAILS page, a Therapist user may select to complete a new evaluation, enter new/additional Physician order information, or add notes regarding any problems experienced by the patient.

The right hand side of the REFERRAL DETAILS page is preferably separated into the second, third, and fourth major areas. As shown in FIG. 6, the top and second area of the REFERRAL DETAILS page preferably includes more specific information about the particular referral, displayed in an easy to read format. This specific information may include the patient name and address, the patient date of birth, the therapy start date, the nature of the referral, the particular joint or joints involved in the injury, the reasons for referral, the name of the referring physician, and any special notes which may have been provided by the referring physician.

The middle and third major area displays a graph detailing results of functional outcomes score results from functional evaluations performed over the course of the therapy. As discussed earlier, functional evaluations are assigned numerical scores or functional outcome once they are completed. Referring to FIG. 6, one can see that this patient had a functional evaluation of his lumbar on Feb. 8, 2005 and the functional score assigned to the outcome was an “8”. Further, one can also see that this patient had a second functional evaluation which was performed on Apr. 29, 2005 and the functional score assigned to that outcome was a “7”. Accordingly, the score decreased over time. The chart shown in the third major area displays this decrease in a very graphical way.

The bottom and fourth major area of the REFERRAL DETAILS page contains detailed information related to the therapy progress for that particular referral. This information preferably includes dates of satisfactory completion of each phase of therapy, listings of functional and non-functional evaluations conducted at the start of and during each phase of the therapy, a listing of all functional outcomes performed over the course of therapy, a listing of any problems which may have been reported by the patient or detected by the Physical Therapist during the therapy, listing of short term goals set by the Physical Therapist over the course of therapy and indications as to whether those goals have been met. It is from this fourth area of the REFERRAL DETAILS page that a Physical Therapist user can access information on prior patient evaluations including the type of each individual evaluation previously performed, the date such evaluation was performed. In addition, the Physical Therapist can view the actual numerical value or functional outcome assigned to each functional evaluation. As will be discussed further hereinafter, this numerical value or functional outcome is helpful in assessing the patient response and progress, as will be discussed in further detail hereinafter.

As shown in FIG. 6, in a preferred embodiment of the present invention, the fourth area of the REFERRAL DETAILS page will include a listing of prior evaluations (functional and non-functional) related to that specific referral. The evaluations contained in the listing are preferably listed in reverse chronological order, with the most recent evaluation appearing at the top of the listing. Each prior evaluation listed will preferably include information on the type of evaluation performed, initials or other indicia indicating the Physical Therapist user who performed the evaluation, and the date the detailed evaluation was performed. Thus, for example, in FIG. 6 it is shown that patient, ALIDA DEBOER had a lower body basic evaluation (non-functional) performed on Dec. 1, 2004 and a detailed lumbar evaluation performed on Dec. 2, 2004. Additionally, a second detailed evaluation was performed on Feb. 8, 2005.

Referring still to FIG. 6, the REFERRAL DETAILS page also includes a listing of all functional outcomes from previously performed functional evaluations. As indicated above, functional evaluations are assigned a score or functional outcome and these scores are listed, as shown in FIG. 6, in a separate listing of functional outcomes. Once again, the numerical value is assigned to the functional evaluations by the system and is based upon the patient's responses to the questions posed when the functional evaluation was conducted. Referring to FIG. 6, one can see that this patient had a functional evaluation of his lumbar on Feb. 8, 2005. Thus, there is an evaluation listed under the evaluations heading for this date. Additionally, the functional outcome or score assigned to this evaluation was an “8”. Thus, there is an entry under the functional evaluations heading for this value/score Further, one can also see that this patient had a second functional evaluation which was performed on Apr. 29, 2005 and the functional score assigned to that evaluation was a “7”. Preferably, each functional outcome listed on the REFERRAL DETAILS page will also include a user selectable link, which when selected, will then display to the user the particulars for that functional evaluation, including the responses to each of the individual questions/observations which were recorded when the functional evaluation was originally performed and the individual scoring for each response.

As described earlier, in a preferred embodiment, the REFERRAL DETAILS page will also include a graphical chart. The graphical chart will show the scores from the functional outcomes in a line graph chart. The chart may also be configured to display the summation of specific active range of motions for each specific joint evaluated in prior evaluations. In this way, a Doctor/Physician or Physical Therapist user accessing the REFERRAL DETAILS page for a particular patient can quickly look to see if both lines in the chart are trending upward, indicating a positive response to the therapy. Alternatively, if both lines in the chart are trending downward then the patient is not responding positively to therapy and further investigation and diagnosis is warranted.

Referring again to FIG. 6, the REFERRAL DETAILS page also allows a Physical Therapist user to enter and track any problems which may be reported by the patient or witnessed by the Physical Therapist user during the course of the therapy. For example, if the Physical Therapist user noted any increased swelling about the knee joint during the course of post operative therapy for a knee injury, the Physical Therapist user can enter such problem from the REFERRAL DETAILS page, including the date of first reporting or observation of the problem. Thereafter, both the Doctor/Physician and the Physical Therapist user will be alerted to the problem every time each retrieves that particular REFERRAL DETAILS page for that particular referral/patient. Once the problem has been resolved, the Physical Therapist user may enter such information, along with the date the problem was resolved. Another main feature of this invention is that it tracks which goals have been met and which have not, it also tracks the goals which have been met, past documentation simply drops a goal once it has been met and you would have to go back to look at which goals have been set and then met or not met. It is the same with the problem list, this software tracks the current problem list along with problems that have been resolved which again speaks to improvements or lack of improvements of a patient.

Evaluations Pages Overview

As explained earlier, one of the key features of the software of the present invention is the standardization of therapy evaluations. As explained, currently patient evaluations are very subjective and depend greatly upon the skill and training of the therapist involved. One main object of the invention is to provide thorough, comprehensive and completely standardized evaluations which will enable all therapists to provide more uniform evaluation, diagnosis and treatment options.

Referring again to FIG. 6, in the first area on the left hand side of the REFERRAL DETAILS page are a number of user navigational items which a user may select in order to perform various tasks. Two such items shown in the listing are the New Evaluation and the New Functional Evaluation. When a Physical Therapist user selects either of these options, a pull down menu of available types of evaluations is displayed from which the Physical Therapist user may select the appropriate evaluation. Accordingly, if the Therapist user selects the New Evaluation option, a listing of all available non-functional evaluations is displayed. Similarly, if the Therapist user selects the New Functional Evaluation option, a listing of all available functional evaluations is displayed. Upon selecting one of the available evaluations from the pull down menu, the Physical Therapist user will be taken to that specific EVALUATIONS PAGE for that evaluation, where the Physical Therapist user may then perform the new and appropriate evaluation.

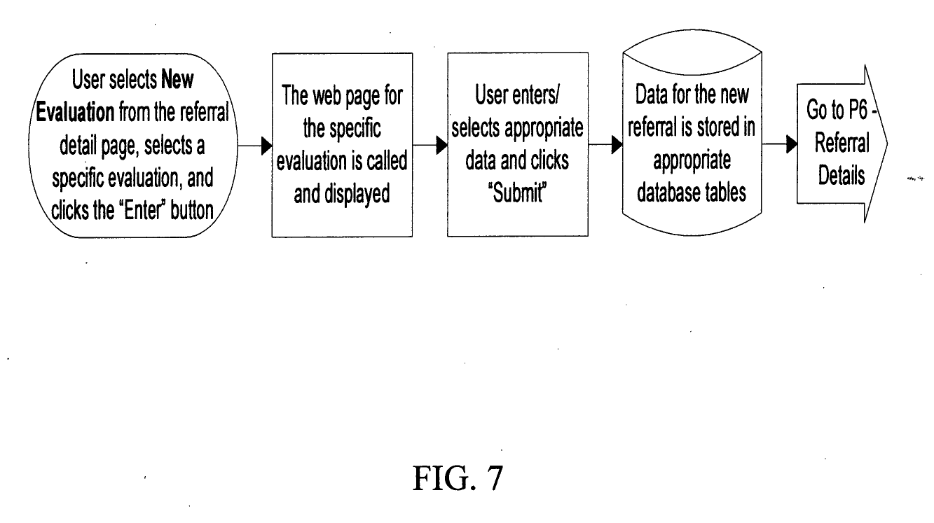

FIG. 7 illustrates a flow chart which shows the steps performed in conducting a new evaluation in accordance with a preferred embodiment of the present invention. As shown in FIG. 7, a Physical Therapist user selects the NEW EVALUATION option from the REFERRAL DETAILS page. Upon selecting this option, a pull down menu is preferably displayed showing all possible non-functional evaluations, thereby allowing the user to select the new type of evaluation to be performed. The Physical Therapist user selects the particular evaluation option he or she wishes to perform and a new EVALUATION PAGE is displayed. The steps are similar for performing functional evaluations except that the Therapist user selects the NEW FUNCTIONAL EVALUATION option from the REFERRAL DETAILS page.

In a preferred embodiment, there are at least fifteen specific and proprietary evaluations that can be documented using the software of the present invention. It is understood that the number of evaluations may expand as needed so long as each evaluation is uniform in its approach and content. Each evaluation has its own particular EVALUATION PAGE which contains specific requested information pertinent to that evaluation. Among the preferably available evaluations preferably included in a preferred embodiment of the present invention are the following:

```
  (1) Ankle - Detailed Evaluation
  (2) Ankle - Functional Evaluation
  (3) Cervical Spine - Detailed Evaluation
  (4) Elbow/Forearm - Detailed Evaluation
  (5) Hand - Functional Evaluation
  (6) Hip - Detailed Evaluation
  (7) Hip - Functional Evaluation
  (8) Knee - Detailed Evaluation
  (9) Knee - Functional Evaluation
(10) Knee, Kujalla Patellofemoral - Functional Evaluation
(11) Knee, Lysholm - Functional Evaluation
(12) Lower Body - Basic Evaluation
(13) Lumbar - Detailed Evaluation
(14) Lumbar - Functional Evaluation
(15) Neck - Functional Evaluation
(16) Shoulder - Detailed Evaluation
(17) Shoulder - Functional Evaluation
(18) Shoulder, Athletic - Functional Evaluation
(19) Upper Body - Basic Evaluation
```

As explained earlier, for each specific evaluation, a user is directed to a new EVALUATION PAGE which has been specifically developed to standardize the evaluation process and capture particular objective and subjective informational elements pertinent to that evaluation. As described with reference to the different types of evaluations which may be performed utilizing the software of the present invention, these evaluations may be functional or non-functional, or any combination of the two. Preferably, a functional evaluation is a series of questions that the patients answer based on their subjective feeling about what caused them to have pain and how much limitation they may feel that they have when doing basic movements. Functional evaluations are often used in research to determine if the patient feels they are improving or getting worse. A non-functional evaluation is preferably comprised of a series of selected objective measurements (range of motion, strength, balance, special tests) to be conducted/measure by the physical therapist. When a patient is truly improving, both the subjective complaints of limitations and pain improve, while their objective measurements also improve. Alternatively, when a patient regresses both the subjective complaints and the objective measurements get worse, which means they need to return to the referring doctor or physician.

An alternate scenario is where either the functional or the non-functional detailed evaluation improves, while the other gets worse. In this case, the functional evaluation may get worse or stay the same while the objective non-functional detailed evaluation measurements may improve. This could signal to the Physical Therapist that either the patient is magnifying their symptoms for some reason, or they do not realize the improvement they have made. Over the last several years, insurance companies require physical therapists to demonstrate improvement on both ends (improved functional levels and improved objective measurements), and the software of the present invention includes simple and concise methods for evaluating and comparing both, on the same page.

When conducting an evaluation, the informational elements are entered by the Physical Therapist user and then stored in a data base format such that the informational elements may then be evaluated and compared with future evaluations as therapy progresses. In a preferred embodiment, each particular EVALUATION PAGE contains a series of subjective questions and/or objective measurements/evaluations which should be completed by the therapist conducting the evaluation. The questions and/or measurements/evaluations have been compiled from an amalgam of various medical literature and medical information available and include several proprietary questions and evaluations, all of which have been specifically tailored and selected in order to standardize and streamline the evaluation process such that evaluations for any one particular ailment or injury can be uniform and will be independent of the particular level of skill, education and experience of the therapist/user.

Moreover, the traditional physical therapy evaluation focuses only on abnormalities, but neglects documenting aspects which appear normal. Utilizing the software of the present invention, the therapist will have the opportunity to document not only abnormalities; but, also normal patient conditions. For example, if a patient comes in for an evaluation but is not wearing a brace or utilizing an assistive device, a physical therapist may or may not note the absence of such items and there may be no mention of it on the evaluation. Utilizing the software of the present invention, the therapist is prompted to answer a question as to whether the patient is wearing a brace or utilizing an assistive device, with the option “none” being available as one of the choices. This is important as the physical therapy chart is a legal document, and only what is documented exists in the future, there for the more information captured about the patient at the time of evaluation the better.

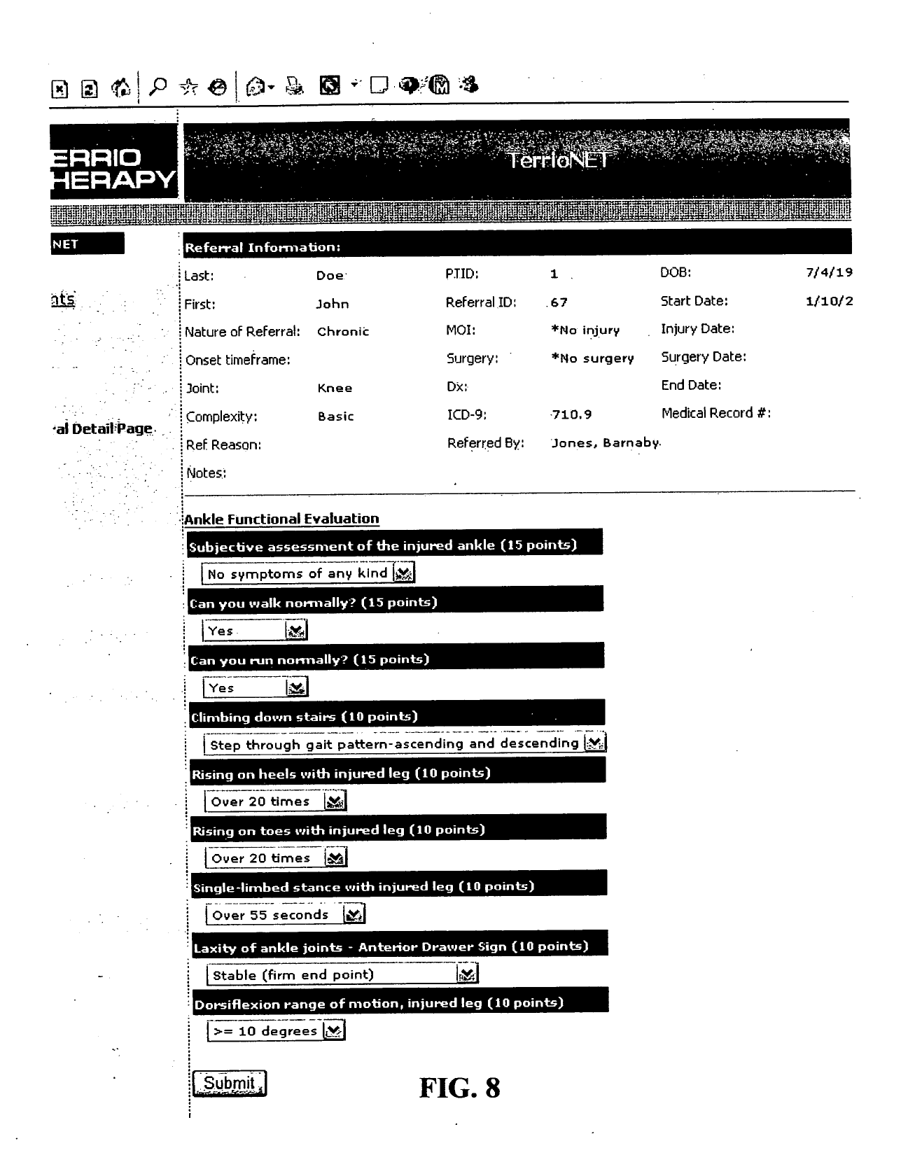

FIG. 8 illustrates a sample EVALUATION PAGE for conducting a functional evaluation of an ankle injury in accordance with a preferred embodiment of the present invention. As shown in FIG. 8, some of the questions (questions 1, 2, 3) contained on the EVALUATION PAGE are directed toward obtaining subjective information from the patient regarding his or her pain, mobility, dexterity and everyday functional ability. With these questions, the Physical Therapist user will ask the patient the question and then provide the patient with the several possible answers. The Physical Therapist will then enter the appropriate answer, as selected and answered by the patient. It is understood that the types, kinds and numbers of subjective questions contained in any specific EVALUATION PAGE may vary dependent upon the particular physical ailment or injury being evaluated.

As further shown in FIG. 8, some of the questions in on the EVALUATION PAGE are also directed toward recording more objective information and recording actual measured observations of the therapist during the evaluation process. For example, they may evaluate the patient's ability to stand on an injured leg (question 7), his or her ability to rise on his or her toes (question 6), and/or his or her ability to perform a particular motion (questions 8 and 9). Once again, it is understood that the types, kinds and numbers of objective questions contained in any specific EVALUATION PAGE may vary dependent upon the particular physical ailment or injury being evaluated.

In a preferred embodiment of the present invention, each individual answer is assigned an evaluation or score and based upon all of the answers to each of the questions, the overall evaluation is assigned a total numerical value or functional outcome. Preferably, the total numerical value or score is simply the sum of all the individual scores assigned to each of the individual answers provided in the evaluation. Alternatively, it may be a weighted average of the responses. In a preferred embodiment, the total numerical value or score is on a scale of 0 to 10, such that a maximum total functional outcome or score would be 10 and minimum total score would be 0. Alternatively the scale may be reversed with 0 being a maximum score and 10 being a minimum score. Use of a total functional outcome or score will be very beneficial in evaluating the progress of the patient as described in further detail hereinafter.

In a preferred embodiment, higher points are assigned to more “positive” responses, observations and evaluations; while lower points are assigned to less “positive” responses. In this way, patients having higher levels of pain, decreased mobility, and less ability to perform simple functions/evaluations, as observed by the therapist, will receive lower points and thus a decreased total numerical value or score. Meanwhile, patients having lower levels of pain or discomfort, increased mobility, and a stronger ability to perform simple functions/evaluations, as observed by the therapist, will receive higher points and thus an increased total numerical value or score. Accordingly, as patients are continuously treated and evaluated over the course of their individual therapy, the goal is to continually increase their total numerical value or score. If the numerical value or score continues to increase over the course of the therapy, as continued evaluations are performed, then the patient is responsive to the proscribed therapy and is noticeably improving. If, however, the numerical value or score decreases or remains fairly constant over the course of the therapy, as continued evaluations are performed, then the patient is non-responsive to the proscribed therapy and is not improving, thereby signaling to the therapist that a deeper problem may exist. Accordingly, the therapist can then discuss the issue with the patient's referring physician and an alternate therapy program may be developed or the patient may be re-examined by the referring physician in order to better ascertain the reason for the continued injury/ailment.

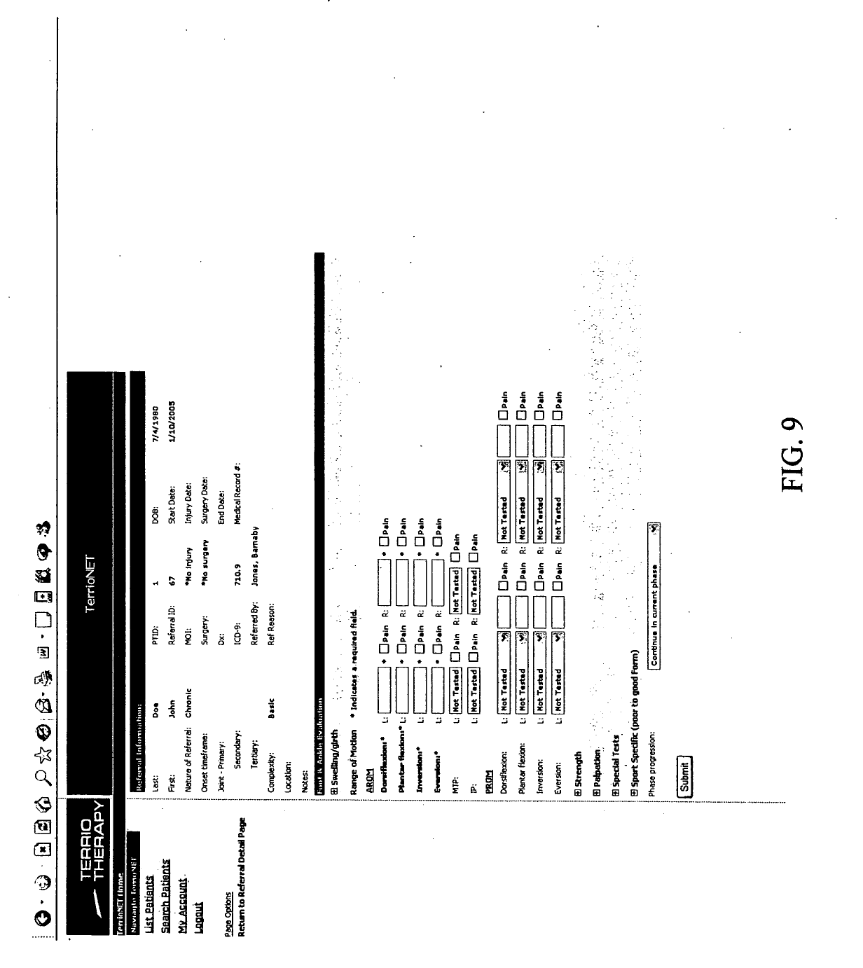

Referring to FIG. 9, there is shown a sample EVALUATIONS PAGE for conducting a detailed evaluation, including objective measurements for range of motion, of an ankle injury in accordance with a preferred embodiment of the present invention. As explained earlier, evaluations in the present invention are either functional or non-functional. In accordance with a preferred embodiment, the software of the present invention will require a Therapist user to conduct both a functional and a non-functional (detailed objective evaluation) of a patient each time he or she is evaluated. Turning to FIG. 9, one can see that completion of this non-functional, detailed evaluation will require the Therapist to make certain objective observations and measurements. The measured results are entered and recorded for future review.

As described earlier herein, the software of the present invention also generates a range of motion value based upon specific mobility and range of motion observed by the Physical Therapist when conducting a non-functional evaluation. Accordingly, the numerical values recorded by the Physical Therapist in FIG. 9, regarding the patient's mobility and range of motion, are utilized in order to come up with a numerical range of motion value. Preferably, the total numerical range of motion value is simply a weighted average of all the individual scores assigned to each of the individual ranges observed during the course of the evaluation. In a preferred embodiment, the range of motion value is on a scale of 0 to 10, such that a patient having a very normal mobility and range of motion would likely score a 10, while a patient having a very limited mobility and range of motion would like score much lower.

As explained earlier, use of a range of motion value will be very beneficial in evaluating the progress of the patient. More specifically, the numerical range of motion value will be graphed separately from the automated scores assigned to functional outcomes of functional evaluations with the ultimate goal of seeing a linear improvement in both scales (active range of motion and functional outcome scores). If both values are trending up then the patient is improving in mobility and range of motion, and is recognizing and responding to this improvement through improved functioning in their every day life. If both values are trending down, then the patient is getting worse and is not responding to the current therapy treatment. If one scale is trending up and the other is trending down (if for example the numerical range of motion value is improving but the overall functional outcome score is not improving) then more investigation is warranted in order to determine why the patient is reporting something different than the objective measurement of active range of motion (i.e. why the patient is not improving functionally in their every day life activity).

Networking Capabilities

The computer-program-implemented system of the present invention for creating and maintaining a patient-doctor-therapist database is described which includes graphic means for receiving patient information, providing patient evaluations, and displaying the patient information and evaluation results as a series of webpages which are interconnected/interlinked. Accordingly, the invention is ideally designed for integration into a network where Doctor/Physician, Physical Therapist and Administrator can all access the information from one source. Preferably, the patient information is stored in a database located on a remote server and is called for display as a user logs into the system and navigates through the system. Software is provided at the location of the Doctor/Physician, the Physical Therapist, and the Administrator such that all three can locally access and edit the patient information, as described previously herein. In this way, there is no need for the Physical Therapist and the Doctor to each keep individual paper files to track the patient's therapy and there is less likelihood of communication error between the two entities. Instead, utilizing the present invention, the Doctor/Physician, the Physical Therapist and an Administrator can all access up to the minute and accurate patient information at any time. This way the Doctor/Physician can accurately track the therapy as it is conducted, view any evaluations conducted by the Physical Therapist (along with any problems encountered and reported by the Therapist) and modify the therapy regime should the same be necessary.

While the description above contains many specifics, these should not be construed as limitations on the scope of the invention, but rather as exemplifications of particular embodiments thereof. One of ordinary skill in the art may make many changes, modifications, and substitutions without necessarily departing from the spirit and scope of the invention. For example, the content and information displayed on the PATIENT DETAILS page as set forth herein is described at a minimum and it is understood that the page may contain additional information presented in any alternate known formats known in the art. Additionally, the invention has been described as having at least fifteen different proprietary evaluations, samples of which have been provided in some detail. It is understood that the number of evaluations may vary and be increased to include different functional, basic and or complete evaluations for additional injuries/ailments to alternate body parts. Accordingly, the scope of the invention should be determined not by the embodiments described above, but by the appended claims and their legal equivalents.

What is claimed is:

1. A computer-program-implemented system for evaluating and monitoring physical therapy patients, the system comprises:
a patient information database for storing detailed patient information;
a graphical user interface for providing the detailed patient information for viewing by a therapist user and a physician, wherein said graphical user interface allows said therapist user to conduct standardized/uniform patient evaluations over the course of therapy and store the results of those patient evaluations in the patient information database such that both the therapist user and the physician can access and monitor the results of those evaluations over a shared network.

2. The computer-program-implemented system of claim 1, wherein the standardized/uniform patient evaluations include function and non-functional evaluations, with a therapist user conducting at least one of each type for each individual patient.

3. The computer-program-implemented system of claim 2, wherein each functional evaluation is assigned a total numerical value or score.

4. The computer-program-implemented system of claim 2, wherein the total numerical value or score assigned to each evaluation is on a scale of 0 to 10, such that as patients are continuously treated and evaluated over the course of their individual therapy, the goal is to continually increase their total numerical value or score as each subsequent functional evaluation is performed.

5. A computer program product for use in providing continued physical therapy to patients, said computer program product comprising:
a computer usable medium having computer readable program code embodied within said medium for causing a computer to:
receive, record and store patient demographic information in a database, said demographic information including the patient name, address, age, injury status, and name of referring physician;
provide a selectable listing of several possible standardized automated evaluations from which a particular standardized automated evaluation may be selected;
displaying a series of questions to be answered in response to said selected standardized automated evaluation;
recording responses to said series of questions and storing said responses to said evaluation in said database for future reference;
assigning a numerical value or score to said evaluation as a function of said recorded and stored responses.

6. The computer program product of claim 5, wherein the computer readable program code embodied within said medium further causes a computer to:
display a PATIENT DETAILS page which includes basic patient demographic information such as the patient's name and address, and further wherein said PATIENT DETAILS page further includes a listing of a series of therapy referrals, each referral in the series including a link to a REFERRAL DETAILS page where more specific information about each such referral can be retrieved.

7. The computer program product of claim 6, wherein the computer readable program code embodied within said medium further causes a computer to:
display a REFERRAL DETAILS page when one of the links associated with one of the referrals in the series is selected, said REFERRAL DETAILS page containing specific detailed information about the selected referral.

8. The computer program product of claim 6, wherein the computer readable program code embodied within said medium further causes a computer to:
display a REFERRAL DETAILS page when one of the links associated with one of the referrals in the series is selected, wherein said REFERRAL DETAILS page includes the therapy start date for such selected referral, the injury date (if known) related to the selected referral, any surgery date (where applicable) related to the selected referral, the name of the referring physician for the selected referral, the particular bones or joints involved in the selected referral, and the complexity of the evaluation.

9. The computer program product of claim 7, wherein the REFERRAL DETAILS page further includes information on prior patient evaluations related to such selected referral, including the type of each individual evaluation previously performed, the date such evaluation was performed, and the numerical value assigned to each such evaluation.

10. The computer program product of claim 5, wherein some of the standardized automated evaluations are functional evaluations which contain questions directed toward obtaining subjective information from a patient regarding his or her pain, mobility, dexterity and everyday functional ability.

11. The computer program product of claim 10, wherein the numerical value or score assigned to a functional evaluation is a functional outcome score based upon specific subjective responses given by the patient when completing the functional evaluation.

12. The computer program product of claim 5, wherein some of the standardized automated evaluations are non-functional evaluations which contain questions directed toward recording more objective information, such as measured observations made during the evaluation.

13. The computer program product of claim 12, wherein the numerical value or score assigned to a non-functional evaluation is a range of motion value which is based upon specific mobility and range of motion observed when conducting the non-functional evaluation.

14. A computer-program-implemented graphical user interface comprising:
an EVALUATIONS PAGE designed to allow a physical therapist to conduct a selected standardized automated evaluation of a physical therapy patient as a function of the patient's injury; wherein said EVALUATIONS PAGE includes:
a series of questions designed to provide a standardized automated evaluation of the patient's injury;
previously recorded responses to said series of questions;
individual numerical values assigned to each of said recorded responses; and
a total numerical value assigned to the standardized automated evaluation, wherein said total numerical value is determined as a function of each of the individual numerical values assigned to each of said recorded responses.

15. The computer-program-implemented graphical user interface of claim 14, wherein some of the questions in the series of questions include in the EVALUATIONS PAGE are directed toward obtaining subjective information from the patient regarding his or her pain, mobility, dexterity and everyday functional ability.

16. The computer-program-implemented graphical user interface of claim 14, wherein some of the questions in the series of questions included in the EVALUATIONS PAGE are directed toward recording more objective information, such as measured observations made during the evaluation process.

17. A method for evaluating and monitoring of patients in physical therapy, said method comprising:
receiving, recording and storing patient demographic information in a database, said demographic information including the patient name, address, age, injury status, and name of referring physician;
selecting a standardized automated evaluation from a previously generated list of several possible standardized automated evaluations;
displaying a series of questions to be answered in response to said selected standardized automated evaluation;
performing said selected evaluation by recording responses to said series of questions and storing said responses to said evaluation in said database for future reference; and
assigning a numerical value or score to said evaluation as a function of said recorded and stored responses.

18. The computer program product of claim 17, wherein some of the standardized automated evaluations are functional evaluations which contain questions directed toward obtaining subjective information from a patient regarding his or her pain, mobility, dexterity and everyday functional ability.

19. The computer program product of claim 18, wherein the numerical value or score assigned to a functional evaluation is a functional outcome score determined as a function of the specific subjective responses given by the patient when completing the functional evaluation.

20. The computer program product of claim 17, wherein some of the standardized automated evaluations are non-functional evaluations which contain questions directed toward recording more
objective information, such as measured observations made during the evaluation.

21. The computer program product of claim 20, wherein the numerical value or score assigned to a non-functional evaluation is a range of motion value determined as function of the specific mobility and range of motion observed when conducting the non-functional evaluation.

22. The method of claim 17, further comprising the steps of:
plotting the assigned numerical value or score to said selected evaluation on a graph made visible to a user;
reselecting a standardized automated evaluation from a previously generated list of several possible standardized automated evaluations, wherein the reselected automated evaluation is preferably the same as that previously selected;
displaying the same series of questions to be answered in response to said reselected standardized automated evaluation;
performing said reselected evaluation by recording responses to said series of questions and storing said responses to said reselected evaluation in said database for future reference;
assigning a new numerical value or score to said re-selected evaluation as a function of said recorded and stored responses; and
plotting the new numerical value or score to said reselected evaluation on the same graph made visible to said user such that said user can see any difference between the original numerical value and the new numerical value in order to monitor a patient's therapy progress.
